(19) United States
(12) Patent Application Publication
(10) Pub. No.: US 2026/0123938 A1
(43) Pub. Date: May 7, 2026

(54) TOURNIQUET WITH OPTIMIZED INNER STRAP AND WINDLASS

(71) Applicant: Composite Resources, Inc., Rock Hill, SC (US)

(72) Inventors: Michael James Casella, Fort Mill, SC (US); Stephen Perkins, Rock Hill, SC (US); Jesse Arnold Fourt, Menlo Park, CA (US); Christopher Steven Hill, San Francisco, CA (US); Daniel Chien-Cheng Huang, Victorville, CA (US); Thomas Kai Wucherpfennig, San Francisco, CA (US)

(21) Appl. No.: 19/380,850

(22) Filed: Nov. 5, 2025

Related U.S. Application Data
(60) Provisional application No. 63/716,614, filed on Nov. 5, 2024.
(60) Provisional application No. 63/893,324, filed on Oct. 3, 2025.
(60) Provisional application No. 63/893,321, filed on Oct. 3, 2025.
(60) Provisional application No. 63/893,317, filed on Oct. 3, 2025.

(51) Int. Cl. A61B 17/132

(52) U.S. Cl. CPC A61B 17/1327

(57) ABSTRACT

A tourniquet for restricting a flow of blood in a body part is provided. A tourniquet may comprise a tourniquet band having an outer sleeve and an inner tightening strap in slidable engagement with the outer sleeve. The inner tightening strap is formed of a twill weave of polymer material to achieve a greater number of warp ends and provides a single routed inner strap with the tensile strength and relatively low elasticity to support the forces needed to apply occlusion pressure to a body part. A portion of the inner tightening strap is anchored to the outer sleeve. A tightening mechanism is provided to tighten the inner strap. A buckle is anchored to the first end of the tourniquet band. The buckle defines an adjustment port adapted for receiving the tail end of the tourniquet band for forming the tourniquet band into a loop.

### RELATED APPLICATIONS

This application claims the benefit of priority under 35 U.S.C. § 119(e) to U.S. Provisional Patent Application 63/716,614, entitled “Tourniquet with Improved Strap Routing,” filed Nov. 5, 2024, U.S. Provisional Patent Application 63/893,317, entitled “Tourniquet with Improved Strap Routing,” filed Oct. 3, 2025, U.S. Provisional Application 63/893,321, entitled “Tourniquet with Overmolded Buckle and Improved Strap Routing,” filed Oct. 3, 2025, and U.S. Provisional Application 63/893,324, entitled “Tourniquet with Optimized Inner Strap and Windlass,” filed Oct. 3, 2025, which are hereby incorporated by reference herein.

### TECHNICAL FIELD

The present disclosure relates to tourniquets. Even more particularly, embodiments of the present disclosure relate to tourniquets with overmolded buckles and improved strap routing.

### BACKGROUND

Uncontrolled blood loss is a leading cause of mortality in emergency scenarios, particularly when the injured individual is alone and immediate medical assistance is unavailable. Tourniquets are widely recognized as effective devices for stemming hemorrhage from extremity injuries in such situations. For solo emergency use, the victim must be able to apply the tourniquet to his or her own arm or leg and occlude blood flow, preferably using only one hand to occlude blood flow.

Prehospital tourniquets are designed to be easily used by first responders or the injured individuals themselves to control severe bleeding before transport to a hospital. Some examples of prehospital tourniquets are described in U.S. Pat. Nos. 10,016,203, 8,888,807, 7,842,067. One notable example is the COMBAT APPLICATION TOURNIQUET (CAT) GEN 7, by Composite Resources Inc., of Rock Hill, South Carolina, USA (all tradenames, trademarks, and the like used herein are the property of their respective owners). This device is widely used by both military personnel and civilian emergency responders.

In many emergency scenarios, including those occurring during recreational activities such as hiking, rock climbing, and camping, the practicality of carrying a tourniquet is influenced by its size and weight. If a tourniquet is excessively bulky or heavy, individuals are less likely to include it in their gear, thereby reducing its availability when needed. Accordingly, there is a need for a compact and lightweight tourniquet that can be conveniently packed, transported, and effectively deployed in remote or austere environments. Additionally, there is a need for tourniquet designs that are compatible with standard equipment holders commonly used by law enforcement and military personnel, such as firearm magazine pouches.

### SUMMARY

The present disclosure provides improved tourniquet systems and related methods. Embodiments may incorporate improved inner strap routing and optimized inner straps and windlasses. Certain embodiments incorporate an overmolded buckle mechanism that facilitates enhanced routing of the inner tensioning strap. Further, the inner tensioning strap may be routed in a manner that minimizes or eliminates the inner tensioning strap tightly weaving through hardware. The improved architecture supports a more compact and efficient tourniquet design, enabling reductions in overall device size without compromising functional performance.

According to one aspect of the present disclosure, a tourniquet comprises a tourniquet band, a tightening mechanism. The tourniquet band comprises an outer sleeve and an inner tightening strap in slidable engagement with the outer sleeve. A portion of the inner tightening strap is anchored to the outer sleeve. As one example, a second end of the inner tightening strap may be anchored to the second end of the outer sleeve. The tightening mechanism is operable to tension the inner tightening strap occlusal pressure. The buckle may be overmolded to one end of the tourniquet strap and provides an adjustment port through which the tail end of the tourniquet band can be passed to form the tourniquet band into a loop. In one embodiment, the inner tightening strap is single pass routed through hardware such as the tightening mechanism. According to one embodiment, the tightening mechanism comprises a windlass.

The buckle may be overmolded to the outer sleeve and the inner tightening strap. The buckle, the outer sleeve and the inner tightening strap may comprise materials of a similar melting temperature to promote thermal fusion during molding. For example, the buckle, the outer sleeve and the inner tightening strap may include nylon.

In one embodiment, a first end of the inner tightening strap extends out of the outer sleeve past the first end of the outer sleeve and the buckle is overmolded to both the first end of the inner tightening strap and the first end of the outer sleeve.

The buckle may include a first lateral portion, a second lateral portion, a first side portion and second side portion extending between the first lateral portion, where the first lateral portion, the second lateral portion, the first side portion and the second side portion form a structural frame that defines the adjustment port, where the first side portion and the second side portion form grip shoulders adjacent to the adjustment port. The first side portion and the second side portion may form a widest part of the buckle. The adjustment port may be located at the widest part of the buckle. The buckle may include a tooth for inhibiting motion of the outer sleeve, the tooth projecting into the adjustment port.

The tourniquet may comprise a plate coupled to the outer sleeve behind the tightening mechanism. For example, the plate may be coupled to the inner side of the outer sleeve. The outer sleeve may define an opening at the plate through which the inner tightening strap is routed to the tightening mechanism. A portion of the outer sleeve, however, may extend from the first end of the plate to the second end of the plate over the side of the plate that faces the windlass.

A securing mechanism may be provided to secure the tightening mechanism. For example, one or more hooked catches may extend outward from the plate. The outer sleeve and inner band run may run between or adjacent to the hooked catches over a portion of the plate.

The tourniquet may include a clip coupled to a first hooked catch from the pair of hooked catches and configured to rotate to engage a second hooked catch from the pair of hooked catches to further secure the windlass. The clip may include a metal wire form and is rotatable between an open position and a closed position. The second hooked catch may include a retaining feature to lock the clip in the closed position.

Another general aspect of the present disclosure includes a method of manufacturing a tourniquet. The method may include a method for manufacturing a tourniquet overmolding a buckle to a first end of a tourniquet band, where the buckle includes an adjustment port for receiving a second end of the tourniquet band to form the tourniquet band into a loop. Overmolding the buckle to the first end of the tourniquet band may include securing a first end of an outer sleeve and a first end of an inner tightening strap in a mold with the first end of the inner tightening strap projecting out of the outer sleeve and overmolding the buckle to the first end of the outer sleeve and the first end of the inner tightening strap. The method may further include attaching a latch plate to the outer sleeve, single-pass routing the inner tightening strap through a windlass for tightening the inner tightening strap, and anchoring a portion of the inner tightening strap to the outer sleeve

Another general aspect of the present disclosure includes a tourniquet for restricting a flow of blood in a body part. The tourniquet may comprise a tourniquet band, the tourniquet band having a tourniquet band first end and a tourniquet band tail end. The tourniquet band may comprise an outer sleeve, the outer sleeve having an outer sleeve first end and an outer sleeve second end; and an inner tightening strap in slidable engagement with the outer sleeve, the inner tightening strap having an inner tightening strap first end and an inner tightening strap second end, wherein a portion of the inner tightening strap is anchored to the outer sleeve, the inner tightening strap comprising a twill weave of polymer threads. The tourniquet may further comprise a windlass operable to tighten the inner tightening strap. The windlass may define an aperture through which the inner tightening strap is single-routed. The tourniquet may further include a buckle anchored to the tourniquet band first end, the buckle defining an adjustment port adapted for receiving the tourniquet band tail end for forming the tourniquet band into a loop.

The twill weave may be formed from threads materials selected such that a single-routed inner tightening strap has a tensile strength and low elasticity to support the forces needed to apply occlusion pressure to a body part. By way of example, but not limitation, the twill weave may be formed of nylon threads or threads of ultra-high-molecular-weight-polyethylene. The inner tightening strap, in some embodiments, may be approximately 1 1/32 inches wide and 0.034 inches thick. In some embodiments, the slot through the windlass has a 47.1%-75.6% clearance,

### BRIEF DESCRIPTION OF THE DRAWINGS

The drawings accompanying and forming part of this specification are included to depict certain aspects of the disclosure. It should be noted that the features illustrated in the drawings are not necessarily drawn to scale. A more complete understanding of the disclosure and the advantages thereof may be acquired by referring to the following description, taken in conjunction with the accompanying drawings in which like reference numbers indicate like features.

FIGS. 1A and 1B are diagrammatic representations of one embodiment of a tourniquet.

FIG. 2 is a diagrammatic representation of a first elevated outer view of a portion of one embodiment of a tourniquet;

FIG. 3 is a diagrammatic representation of a side view of a portion of one embodiment of a tourniquet;

FIG. 4 is a diagrammatic representation of an elevated inner view of a portion of one embodiment of a tourniquet;

FIG. 5 is a diagrammatic representation of a second end of one embodiment of a tourniquet;

FIG. 6 illustrates one embodiment of a buckle end of a tourniquet;

FIG. 7 illustrates cross-sectional view of one embodiment of a buckle end of a tourniquet; and

FIG. 8 is a flow chart illustrating one embodiment of a method for manufacturing a tourniquet.

### DETAILED DESCRIPTION

The disclosure and various features and advantageous details thereof are explained more fully with reference to the exemplary, and therefore non-limiting, embodiments illustrated in the accompanying drawings and detailed in the following description. It should be understood, however, that the detailed description and specific examples, while indicating the preferred embodiments, are given by way of illustration only and not by way of limitation. Descriptions of known techniques may be omitted so as not to unnecessarily obscure the disclosure in detail. Various substitutions, modifications, additions and/or rearrangements within the spirit and/or scope of the underlying inventive concept will become apparent to those skilled in the art from this disclosure.

Before proceeding, some additional context may be helpful. As discussed, there is a desire for smaller, more conveniently carried tourniquets that fit in existing equipment holders, or in new storage or transportation locations not previously feasible with existing tourniquet sizes. However, efforts to reduce the physical dimensions of conventional tourniquet designs encounter several functional limitations. First, it is preferable that the constriction band maintain a minimum width to achieve effective vascular occlusion; industry standards generally recognize 1.5 inches as the minimum acceptable width to achieve effective occlusion pressure (e.g., the CAT GEN 7 tourniquet utilizes a 1.5-inch-wide band). Moreover, the band is preferably long enough to accommodate the limb circumference of a broad user population. For instance, the 99th percentile leg circumference is approximately 30.7 inches, necessitating a band length that exceeds this measurement to ensure universal applicability (e.g., the CAT GEN 7 features a 37.5-inch-long band). Consequently, while there is a clear desire to reduce the overall package size of tourniquets, it remains preferable that the band dimensions—both in width and length—remain consistent with established designs to preserve efficacy.

Additionally, the hardware of prior designs cannot simply be miniaturized to achieve a smaller package size as doing so would result in high torques. Prehospital tourniquets typically utilize a dual-component band structure comprising an outer sleeve and an inner tensioning strap. The inner strap is anchored within and slidable relative to the outer sleeve. The inner strap emerges from the outer sleeve and is routed through a windlass mechanism to facilitate incremental tightening. In many designs, including the CAT GEN 7, the inner strap forms a continuous loop within the outer sleeve and is double-routed through various hardware slots—passing through the slots twice. Specifically, the strap extends from the free end of the outer sleeve to the buckle, loops through a slot in the buckle, and returns to the free end, where both ends are secured. This configuration forms a loop and results in two overlapping inner strap layers. Both layers of the inner strap emerge from the outer sleeve to pass through the windlass, effectively doubling the strap's passage through the windlass slot.

For example, the end buckle of the CAT GEN 7 includes a first lateral end, a second lateral end, and an intermediate lateral bar that, in cooperation with the sidewalls of the buckle, form two slots, which can be referred to as an attachment slot and an adjustment slot. The adjustment slot is the slot through which the free end of the tourniquet band is threaded to form a loop and tighten the tourniquet band about part of the victim's body. The attachment slot is used to anchor the buckle to the outer sleeve of the tourniquet band and the adjustment slot is for creating a loop and tightening the tourniquet about an object. One end of the outer sleeve passes through the attachment slot of the buckle and is attached back to itself to create an eye at which the buckle is thus anchored. The inner strap extends from the free end of the outer sleeve to the buckle, loops through an attachment slot in the buckle, and returns to the free end, where both ends are secured to form a continuous loop.

The windlass is typically positioned above a base plate that rests against the victim when the tourniquet is applied. The base plate includes narrow strap routing slots—similar to the slots of a tri-glide slide—designed to secure the base plate to the strap and retain the position of the base plate on the strap. In the CAT GEN 7, for example, the base plate incorporates dual-slot arrangements, resembling tri-glide slides, at both ends. At the end of the base plate toward the free end of the tourniquet band, the tourniquet band (outer sleeve and both layers of the inner band) passes from the front to the rear of the base plate through one slot and reemerges through the adjacent slot, weaving tightly behind a plastic bar. A similar configuration exists at the opposite end, between the windlass and the buckle, where the tourniquet band (outer sleeve and both layers of the inner band) again weaves tightly behind a plastic bar. Because the inner strap is double-routed, the inner strap passes through each of these tri-glide slide-type arrangements twice (e.g., once running from the tip of the band to the buckle and once on the return from the buckle to the tip of the tourniquet band).

Weaving the tourniquet band through the strap routing slots can cause significant friction that inhibits the sliding of the inner strap relative to the outer sleeve as the windlass is turned. This friction can require high windlass torque to overcome. Further, with some tourniquets, the user must turn a windlass several turns before the tourniquet begins building pressure around the body part. Some tourniquets also have a pressure function where, once pressure starts to build, it builds quickly for the first several turns, then flattens out, and then builds sharply, making it difficult for the user to keep turning the windlass.

Reducing the dimensions of prior designs would significantly increase the torque required to operate the windlass mechanism, thereby impairing the tourniquet's effectiveness. For example, simply miniaturizing the hardware, such as reducing the size of the base plate of prior designs would exacerbate the friction caused by weaving the tourniquet band through the slots thereby requiring significantly higher torque to operate the windlass. Moreover, a shortened windlass rod offers reduced mechanical leverage, making it difficult to generate the necessary torque for proper arterial occlusion. Consequently, simply downsizing existing tourniquet hardware designs would compromise their ability to deliver reliable and effective pressure across a broad user population.

Embodiments of the present application provide tourniquets with improved inner strap routing and complementary features that enable a reduced overall size without compromising performance relative to prior prehospital tourniquets. In some embodiments, the inner strap is single-routed (single pass) such that the inner strap is routed through the slots of one or more pieces of hardware once. For example, the inner strap may be routed such that it passes through a slot in the windlass only once. Single-pass routing of the inner strap can allow the tourniquet to be smaller by reducing the doubling up of the inner strap.

Further, in some embodiments, the improved inner strap routing reduces or eliminates the need for the working segment of the inner strap to be woven through strap routing slots in the base plate, thereby reducing hardware-induced friction. This reduction in hardware-induced friction facilitates smoother strap movement during windlass activation, thereby lowering the torque required to achieve effective tightening and allowing hardware components to be smaller. Furthermore, by eliminating pinching of the inner strap at the strap routing slots, embodiments promote more uniform strap motion, which in turn mitigates sudden pressure spikes and torque surges.

Thus, embodiments of the present disclosure can maintain a consistent pressure profile while requiring lower baseline torque and minimizing sudden torque spikes. Such characteristics enhance usability, particularly for individuals with limited strength—such as those self-administering the device—or for users with minimal training, thereby improving the overall accessibility and effectiveness of the tourniquet in emergency scenarios.

In one embodiment, the tourniquet buckle is integrally overmolded onto the terminal end of both the outer sleeve and the inner tightening strap, thereby securely anchoring the strap without necessitating a return loop configuration. By overmolding the buckle to the strap end, the working segment of the inner strap avoids the need for an attachment slot, eliminating the need for the buckle to include a bar for the strap to bend around, thereby promoting the use of a smaller buckle.

Additionally, other components, such as latch plates, may be affixed to the tourniquet band via overmolding, sonic welding, thermal bonding, or other coupling techniques. In certain embodiments, the inner strap does not traverse slots within the latch plate. For example, latch plate configurations may omit tri-glide slide-type slots or similar hardware based routing features, thereby reducing both the spatial footprint required for such hardware and the frictional resistance they introduce. This contributes to a more compact and efficient design, enhancing ease of use and mechanical performance.

Further, prior prehospital tourniquets used double-routed, plain weave inner straps. Embodiments of present disclosure may use a slightly thicker, single-routed twill weave. Although the inner strap may, in some embodiments, be slightly thicker than prior inner straps, the single layer is thinner than two layers of the prior strap. For example, two layers of the CAT GEN 7 inner strap is approximately 0.047″, whereas some embodiments of the present disclosure may have an inner strap with a thickness of approximately 0.034″. Moreover, the twill weave pattern results in a smoother pressure profile.

In non-limiting embodiment, the slot height of the slot through windlass 52 has a 47.1%-75.6% clearance, where the percentage clearance=(slot height−inner strap thickness)/(inner strap thickness). For example, in an embodiment with a 0.034″ thick inner strap, the windlass slot width is 0.05″to 0.0597″, though other slot widths may be used.

The strap routing and design can allow for an overall less bulky inner strap that achieves similar or better performance characteristics compared to prior double routed inner straps. The use of single pass routing allows the tourniquet to be folded into a smaller package size compared, for example, to the CAT GEN 7.

The improved inner strap routing of some embodiments improves the pressure build function of the tourniquet (e.g., lower torque and finer tuning) by reducing friction in the system. With a smaller torque requirement, hardware such as the buckle, latch plate, and windlass can be smaller compared, for example, to the CAT GEN 7.

Thus, single-pass routing of the inner strap and other features discussed herein allow for the use of smaller or reduced hardware creating a smaller package size when stored. For example, the CAT Gen 7 has a 37.5 inch long tourniquet band with a 1.5 inch wide outer sleeve with 1 inch wide inner strap that is 0.0235 inches thick (0.047 inches thick when double routed). An embodiment of a tourniquet according to the present disclosure having a 37.5 inch long tourniquet band with a 1.5 inch wide outer sleeve and a 1 1/32 inch wide (+/− 1/32 of an inch) inner strap that is 0.034 inches thick can be coiled into a package size for storage/transport that is 30% smaller than the package size of the CAT Gen 7 while predicted to achieve similar or improved performance. This allows, for example, the tourniquet to fit in some sizes of standard equipment holders worn by law enforcement and military personnel (e.g., in firearm magazine holders). Further, with the tourniquet band cinched about a body part, the windlass can begin to build pressure within the first turn and continuously build pressure as it is turned.

Further, embodiments may include a robust strap retention clip formed of metal or similar strong material. Such a retention clip may be easier to manipulate and secure compared with a hook and loop strap, while also being less likely to catch on or scratch outside materials such as the pockets of law enforcement uniforms

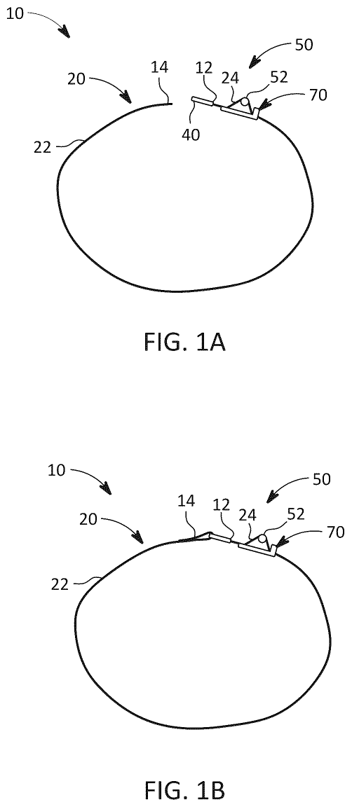

With reference to FIG. 1A and FIG. 1B one embodiment of a tourniquet 10 comprises a band 20, a buckle 40, a tightening mechanism 50, and a securing mechanism 70. A first end of band 20 is anchored to a buckle 40. The tail end of band 20 can be routed through buckle 40 and secured back to tourniquet band 20 to form a loop (FIG. 1B) about a body part.

A tightening mechanism 50 is provided to tighten an inner tightening strap 24. According to one embodiment, tightening mechanism 50 comprises a windlass 52 through which inner strap 24 passes. Rotating windlass 52 causes inner strap 24 to tighten thereby causing tourniquet 10 to apply circumferential pressure to the body part. Securing mechanism 70 includes features to prevent windlass 52 from unwinding.

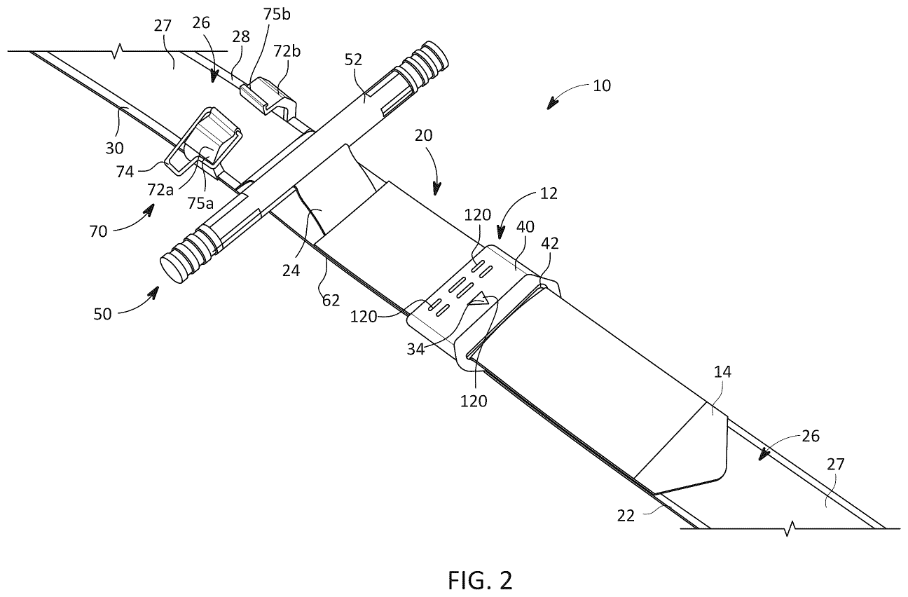

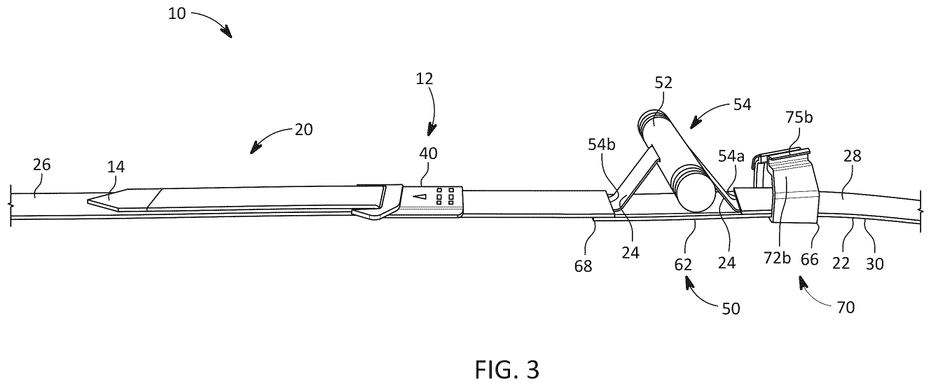

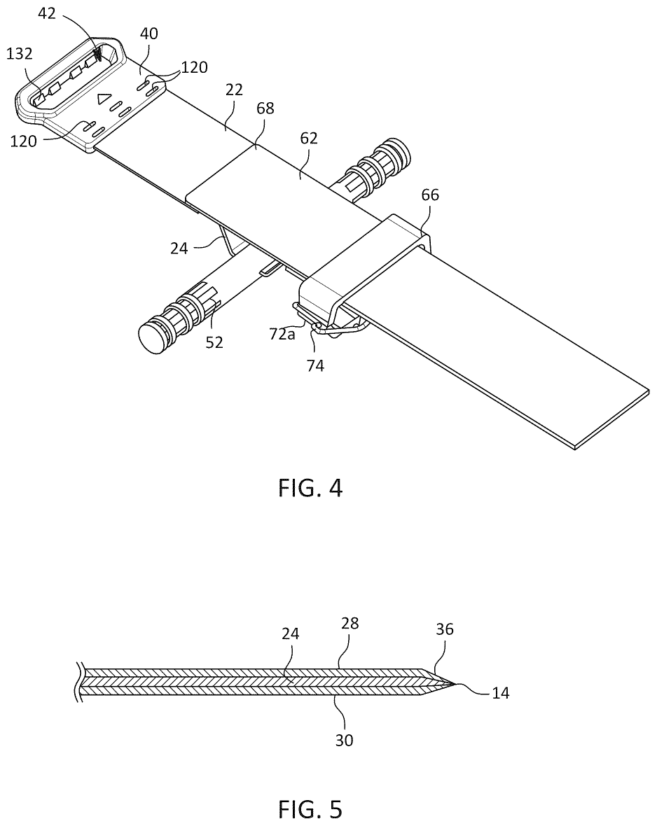

With reference to FIGS. 2-5, FIG. 2 is a diagrammatic representation of a first elevated outer view of one embodiment of a portion of tourniquet 10, FIG. 3 is a diagrammatic representation of a side view of one embodiment of a portion of tourniquet 10, and FIG. 4 is a diagrammatic representation of an elevated inner view of one embodiment of a portion of tourniquet 10, and FIG. 5 is a diagrammatic representation of a second end of one embodiment of a tourniquet.

Tourniquet 10 comprises a tourniquet band 20, a buckle 40, a tightening mechanism 50 and a securing mechanism 70. Tourniquet band 20 comprises an outer sleeve 22 and an inner strap 24. Outer sleeve 22 comprises a longitudinally extensive material having a first end 12 coupled to buckle 40 and a second end 14 (a tail end). When tourniquet 10 is applied to a victim's body, the second end 14 of outer sleeve 22 is looped through adjustment port 42 of buckle 40 and pulled tight, thus providing a means for circumferentially surrounding or encircling a portion of the victim's body.

In accordance with one embodiment, outer sleeve 22 is formed of one or more panels of materials such as, but not limited to, flat weave nylon binding tape. In the illustrated embodiment, sleeve 22 comprises a first panel 28 (e.g., an outer panel) and a second panel 30 (e.g., an inner panel). The edges of panels 28 and 30 are connected, as for example, by sewing, glueing, stapling, clamping, or heat/ultra-sound (sonic) welding, or combinations thereof to form an interior area (e.g., a pocket). In another example embodiment, outer sleeve 22 is formed of a single piece of material, such as by way of example and not limitation, a piece of material that is folded over and seamed to form the interior area.

Band 20 includes fastening mechanisms so that band 20 can be secured to itself after being looped through buckle 40. In one embodiment, the outer surface 26 of outer sleeve 22 includes both hook and loop structures 27. More preferably, outer surface 26 comprises both hook structures and loop structures along substantially the entire length of outer sleeve 22 between the second end of outer sleeve 22 and an opening where inner strap 24 is exposed. Thus, when the second end of outer sleeve 22 is looped through buckle 40, the outer surface 26 may be applied to itself, thereby securing the position of outer sleeve 22. By way of example and not limitation, the outer surface of outer sleeve 22 comprises a length of OMNI-TAPE® (Velcro Industries B.V., Amsterdam, Netherlands), wherein the fastening surface comprises both hook and loop structures on the outer surface 26. The use of a combination of both hook and loop structures on the outer surface 26 of outer sleeve 22 provides the advantage of the tourniquet being quickly adjustable when in use to accommodate a variety of size appendages, as for example, from a person's thigh to a person's forearm. In addition to or in the alternative to using hook and loop material as a fastener, outer sleeve 22 may be fitted with other types of fasteners, such as, but not limited to rigid hooks and loops, buttons, snaps, transverse straps, or other fastening mechanisms.

In use, to size the tourniquet to an area of the victim's body, such as a limb, the user, loops the second end 14 of the outer sleeve 22 through the buckle 40 to form a loop, pulls the tourniquet reasonably tight about the subject body part by pulling on the tail end of outer sleeve 22, and then secures the tail end of outer sleeve 22 to the loop—for example be pressing surface 26 together to interlock hook and loop structures of the outer surface 26 within the region where the outer surface 26 overlaps beyond the buckle 40.

Outer sleeve 22 forms an opening 54 through which inner strap 24 emerges for engagement with windlass 52. For example, first panel 28 may be discontinuous proximate to the tightening mechanism 50, thereby allowing inner strap 24 to be routed through windlass 52. Tightening mechanism 50 can be actuated to tension the inner strap 24 to develop a tension force in the inner strap 24, generating a controlled constriction force suitable for effective vascular occlusion.

Inner strap 24 comprises a length of material extending from a first end 34 and a second end 36 (FIG. 5. In one embodiment, the first end 34 of inner strap 24 is anchored to buckle 40 and a portion of inner strap 24 away from buckle 40 is anchored to outer sleeve 22 as, for example, by sewing, glueing, stapling, clamping, or heat/ultra-sound (sonic) welding, or another anchoring mechanism. According to an even more particular embodiment, only the second end 36 of inner strap 24 is anchored to outer sleeve 22 (for example, at the second end 14 of outer sleeve 22 distal from buckle 40) (FIG. 5) and the remainder of inner strap 24, including first end 34, is not anchored to outer sleeve 22. In other embodiments, the second end of inner strap 24 or other portion of inner strap 24 is anchored to outer sleeve 22 at another location.

Inner strap 24 has a tensile strength and relatively low elasticity to support the forces needed to apply occlusion pressure to a body part. In one embodiment, inner strap 24 is formed from a length of polyester twill weave material. In another embodiment, inner strap 24 comprises a length of ultra-high-molecular-weight polyethylene fabric, such as DYNEEMA webbing. According to one embodiment, inner strap 24 is a length Nylon webbing with a twill weave.

A twill weave with added warp ends allows for more density of material. This allows for a faster, more robust knot build while also reducing the size of the tourniquet. In one example embodiment, inner tightening strap 24 is formed using 840 denier nylon T6 warp thread, 420 denier nylon T6 fill thread, and 75 denier nylon lock thread in a twill weave pattern that has 108 warp ends to produce a strap that is 1 1/32 inches (+/− 1/32 inch) wide and 0.034 inches thick. Other materials, threads and weave patterns may also be used.

Inner strap 24 preferably has frictional characteristics allowing it to slide within the interior space of the outer sleeve 22 when a tensile force is applied to the inner strap 24. The interior space of the outer sleeve 22 may include a substance, such as a powder or other lubricant or a sheet of plastic or other relatively low friction material, to assist with the frictional characteristics between the surfaces of the inner strap 24 and outer sleeve 22.

In accordance with certain embodiments, the inner strap 24 exits the outer sleeve 22 at opening 54 (e.g., through aperture 54a), where it interfaces with the tightening mechanism 50. In the illustrated embodiment, the tightening mechanism comprises a windlass 52, which is preferably fabricated from a polymeric material, although alternative materials may also be employed, and inner strap 24 is routed from the internal area of the outer sleeve 22, through a slot or similar opening in the windlass 52, and subsequently reenters the sleeve's interior (e.g., through aperture 54b).

In some embodiments, the routing of inner strap 24 from aperture 54a to windlass 52 may be frictionless in that inner strap 24 does not weave or bend around any hardware that causes friction. Similarly, the routing of inner strap 24 from the aperture of windlass 52 to aperture 54b may be frictionless in that inner strap 24 does not weave or bend around any hardware that causes friction.

When a user turns windlass 52, windlass 52 pulls inner strap 24 and builds a coil. This action increases tension within inner strap 24, thereby causing the constriction necessary for effective pressure application. According to one embodiment, two close apertures 54a, 54b are spaced and sized to help guide inner strap 24 to windlass 52 and minimize slack in inner strap 24. The dimensions and placement of apertures 54a and 54b are selected to guide the strap efficiently toward the windlass 52, ensuring unobstructed movement of inner strap 24 while reducing the likelihood of binding. This configuration allows the windlass to begin coiling the strap and applying pressure without requiring multiple preliminary turns, thereby facilitating a smooth and progressive buildup of occlusion force. As will be appreciated, if apertures 54a, 54b are too large, inner strap 24 may bind as the coil is wound or released. Thus, the size (length and width) of apertures 54a, 54b can be selected to prevent binding, inhibit coil propagation along band 20, and support reliable resetting of the device.

Tourniquet 10 preferably includes a latch plate 62 behind windlass 52. Preferably, latch plate 62 is flexible enough to bend around the body part as tourniquet 10 is tightened to achieve an occlusion pressure.

Latch plate 62 may include a securing mechanism for securing windlass 52 after tightening. In the illustrated embodiment, securing mechanism 70 comprises a pair of hooked catches 72a, 72b that extend outwardly from latch plate 62 at the first end 66 and are set substantially transverse to the longitudinal axis of tourniquet 10. Latch plate 62 and hooked catches 72a, 72b may be formed of any suitable materials, such as a hard plastic (for example, but not limited to, a Nylon injected molded plastic), metal, composite material or other materials. In some embodiments, the latch plate 62 is formed of a KYDEX® (Kleerdex Company, LLC, Mount Laurel, N.J.) thermoplastic or moldable plastic type of material (e.g., as by injection molding).

Hooked catches 72a, 72b are preferably sized to cup or hold the windlass, or a portion thereof, and prevent it from unwinding. Accordingly, hooked catches 72a, 72b are sufficiently stiff to provide adequate resistance against the tensile force within inner strap 24, as transferred to the hooked catches 72a, 72b by the windlass 52. With two hooked catches, the user can rotate windlass 52 in either direction to apply pressure. When the user has rotated the windlass to apply sufficient pressure to the body part, the user can hook the end of the windlass 52 under the hooked catch 72a, 72b that will prevent windlass 52 from unwinding.

While two hooked catches 72a, 72b are illustrated, other embodiments may use a single hooked catch. For a single hooked catch, the user rotates the windlass in the proper direction to allow the tension in the inner strap 24 to be resisted by the single hooked catch once winding of the windlass and tensioning of the inner strap 24 is completed.

Embodiments may include structures to further secure windlass 52. These structures can allow the user to secure the windlass 52 and move about (or be moved by another person) with less concern of the windlass 52 dislodging from the hooked catches 72a, 72b and unwinding. In one embodiment, tourniquet 10 comprises a rigid clip 74, such as is formed of metal or plastic. In an even more particular embodiment, clip 74 is a metal wire form clip.

Clip 74 is coupled to hooked catch 72a and is configured to rotate into engagement with hooked catch 72b. Hooked catch 72a may incorporate a retaining feature 75a, such as a longitudinal ridge or other detent or feature, to hold clip 74 in its fully open position and prevent premature closure of clip 74. Upon application of a sufficient force, the resilience of clip 74 or hooked catch 72a allows clip 74 to release from the retaining feature 75a and rotate to a closed position. When closing clip 74, the user rotates clip 74 from its initial position to hook clip 74 over hooked catch 72b. This engagement may include clip 74 snapping into a retaining feature 75b, such as a notch, groove or other indent or being captured by a tooth or other detent to secure clip 74 in the closed configuration. In some embodiments, hooked catch 72b includes a lead-in or other feature to help guide hook clip 74 into the correct position.

Other mechanisms may be used to help secure windlass 52, such as, but not limited to, a transversely oriented strap having hook and loop fastening portions or an elastic band that hooks over hooked catch 72a and hooked catch 72b. Helpfully, a user may rest the windlass in one of the hooked catches without deploying the structure that further secures the windlass, conveniently allowing the user to further tighten the tourniquet by unhooking the windlass, rotating the windlass, and re-hooking the windlass. Furthermore, the structures that further secure the windlass may be locked or unlocked one-handed, increasing the circumstances in which a user can lock the structure or increase tension on an already-locked tourniquet, for either a self-application or application to a different victim.

The second panel 30 of the outer sleeve 22 may extend over at least a portion of the latch plate 62 and to buckle 40. In the illustrated embodiment, for example, panel 30 extends from the first end 66 of latch plate 62 to the second end 68 of latch plate 62 over the side of latch plate 62 that faces windlass 52 and continues to buckle 40. Latch plate 62 may be connected to second panel 30, as for example, by overmolding, sewing, glueing, stapling, clamping, or heat/ultra-sound (sonic) welding. In some embodiments, a portion of latch plate 62 extends into the interior space of outer sleeve 22.

First panel 28 may extend through securing mechanism 70, such as between hooked catches 72a, 72b, and is open over at least a part of latch plate 62 to create opening 54 under windlass 52. First panel 28 resumes proximate to the second end 68 of latch plate 62 and extends to buckle 40. The edges of first panel 28 and second panel 30 between the buckle 40 and the second end 68 of the latch plate 62 are preferably connected, as for example, by sewing, glueing, stapling, clamping, or heat/ultra-sound (sonic) welding.

In certain embodiments, the latch plate 62 is integrally overmolded onto the outer sleeve 22. During the manufacturing process, tourniquet band 20 is positioned and clamped within a mold, ensuring accurate alignment and stability. Thermoplastic material is then injected into the mold to form the latch plate 62 along with the hooked catches 72a and 72b. Preferably, the selected plastic exhibits a melt temperature compatible with the second panel 30 of the outer sleeve 22, thereby promoting a robust thermal bond between latch plate 62 and second panel 30. The materials selected for latch plate 62 and second panel 30 of outer sleeve 22 may possess similar melt temperatures to facilitate cohesive bonding during the molding process. For instance, all components may be fabricated from or incorporate nylon, enabling thermal fusion and enhancing the mechanical integration of latch plate 62 with outer sleeve 22.

According to one embodiment, the inner strap 24 does not traverse slots within the latch plate 62. Because latch plate 62 is overmolded or otherwise attached to second panel in a manner that does not require routing slots, latch plate 62 can omit tri-glide slide-type slots and similar routing features, thereby reducing both the spatial footprint required for such hardware and the frictional resistance they introduce. This contributes to a more compact and efficient design, enhancing ease of use and mechanical performance. However, other embodiments may incorporate features such as routing slots and other routing features as part of a latch plate or in other components.

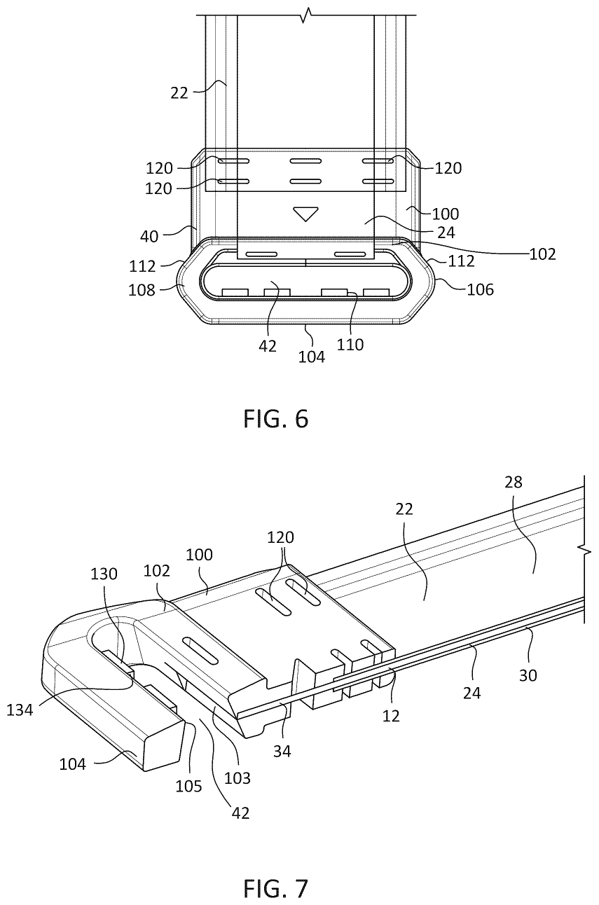

With further reference to FIGS. 6-7, FIG. 6 is a diagrammatic representation of a terminal end of tourniquet band 20 with buckle 40 made transparent to better illustrate and example arrangement of outer sleeve 22 and inner strap 24 in buckle 40 and FIG. 7 is a diagrammatic representation of a cross-sectional view of the terminal end of tourniquet band 20. Buckle 40 comprises a body 100 that is overmolded directly onto the first (terminal) end of tourniquet band 20. During the molding process, the first end 12 of outer sleeve 22 and the first end 34 of inner strap 24 are positioned in the mold and secured by clamping features. These features are configured to prevent displacement or misalignment of the outer sleeve 22 and inner strap 24 during plastic injection.

The molding material is introduced at a temperature calibrated to promote molecular bonding between the injected plastic and the tourniquet band materials. The materials selected for the buckle 40, outer sleeve 22, and inner strap 24 may possess similar melt temperatures to facilitate cohesive bonding during the molding process. For instance, all components may be fabricated from or incorporate nylon, enabling thermal fusion and enhancing the mechanical integration of buckle 40 with both the inner strap 24 and outer sleeve 22.

In some embodiments, the first end 34 of inner strap 24 terminates in inner strap 24 or at the same point as the first end 12 of outer sleeve 22. In other embodiments, the first end 34 of the inner strap 24 extends beyond the first end 12 of the outer sleeve 22, allowing the mold to independently clamp each component. This configuration yields a more robust bond compared to designs in which the first end 34 of inner strap 24 does not project from the sleeve.

Upon removal from the mold, buckle 40 may exhibit shaped openings 120 corresponding to the locations of the clamping features used during the molding process (not all openings are labeled). These openings may be intentionally designed with aesthetic contours or configured to convey information, such as alphanumeric characters or symbols, enhancing both functionality and visual appeal.

Buckle 40 comprises a first lateral portion 102 that defines a transverse wall 103 oriented perpendicular to the longitudinal axis of the tourniquet. Spaced from this segment, a lateral bar 104 defines a second transverse wall 105. Buckle 40 further includes a first side portion 106 and a second side portion 108, each extending between respective ends of the lateral bar 104 and the lateral portion 102, thereby forming a structural frame. Adjustment port 42 is defined between the lateral portion 102 and the lateral bar 104, serving as a passageway for the outer sleeve 22 to loop through the buckle 40 during application and release of the tourniquet around part of the victim's body, such as a limb.

Teeth configured to inhibit movement of the outer sleeve 22 with respect to the buckle 40. may project into adjustment port from lateral portion 102 or lateral bar 104. In the illustrated embodiment, lateral bar 104 includes tooth set 110. Alternatively, or in addition, the lateral portion 102 may also feature a corresponding tooth set. A tooth set comprises at least one tooth, although additional teeth may be included to enhance retention. Each tooth may include an outer surface 130, an inclined surface 132 (FIG. 4), and an edge or projection 134 therebetween.

During operation, the second end 14 of the outer sleeve 22 is routed through adjustment port 42 from the inner side (i.e., the side oriented toward the victim's body), passing over lateral bar 104. Once tourniquet band 20 has exited the adjustment port 42 and been tensioned to the desired degree around the victim's body (e.g., around a limb), a fastening mechanism such as hook-and-loop material may be employed to secure the outer sleeve 22 back onto itself. This configuration ensures reliable strap retention while facilitating rapid and intuitive adjustment during emergency application.

Side portions 106 and 108 of buckle 40 extend laterally outward to form pronounced grip shoulders 112. These shoulder features are strategically positioned adjacent to the adjustment port 42, which is located at or near the widest section of the buckle. The grip shoulders 112 serve as tactile reference points, enabling users to easily locate and identify the buckle by feel alone—particularly in low-visibility or high-stress environments. Additionally, these extended surfaces provide enhanced gripping areas, allowing users to securely grasp and exert pulling force on the buckle during manipulation. This ergonomic design facilitates intuitive handling and improves operational efficiency, especially when rapid deployment or adjustment of the tourniquet is required.

For example, grip shoulders 112 bump laterally outward to be wider than the remainder of buckle 40, latch plate 62, hooked catches 72a, 72b and tourniquet band 20. With the tourniquet 10 folded, the user can feel for grip shoulders 112 to easily grab and pull buckle 40 by shoulders 112. Further, the user can easily feel where the buckle is when stretching the tourniquet out or wrapping the tourniquet around a limb (or other body part) when the user cannot see buckle 40.

In one embodiment, tourniquet 10 is stored in a folded-up configuration with the tail end of band 20 already through buckle 40 and secured to the outer surface of the band to form a loop. In another embodiment, if the tail end of band 20 is free, the user loops the free end of band 20 through adjustment port 42 of buckle 40 to create a loop and secures the tail end to the outer surface of band 20 using the hook and loop material. The user can then slide the preformed loop over a body part (e.g., a limb), unsecure the tail end of band 20, pull tail end of band 20 to achieve an initial cinch, and resecure band 20 to itself.

In other cases, it may not be possible or optimal to use a preformed loop. For example, if a limb is pinned, it may not be possible to slide a preformed loop over the limb. In such a case, the user may undo the preformed loop if present, thread the free end of band 20 of the tourniquet around the body, route the free end of band 20 through adjustment port 42 of buckle 40 to create a loop around a body part, pull the free end to tighten the loop around the body part and secure band 20 to itself.

Once the initial cinch is achieved and band 20 secured to itself, the user winds windlass 52 to tighten inner strap 24, thereby causing the tourniquet to apply circumferential pressure to the body part. When a sufficient pressure is achieved, the user hooks the end of the windlass 52 under hooked catch 72a or hooked catch 72b and locks clip 74.

As discussed, tourniquet buckle 40 may be integrally overmolded onto the terminal end of outer sleeve 22 and inner strap 24, thereby securely anchoring inner strap 24 without necessitating a return loop configuration. The inner strap 24 can be single-pass routed through the windlass 52 mechanism, with the first end 34 of inner strap 24 fixed at buckle 40. The use of single pass routing can allow the tourniquet to be folded into a smaller package size compared, for example to the CAT GEN 7.

Further, by overmolding the buckle to the strap end, the working segment of the inner strap 24 that is tensioned by windlass 52 avoids the need to bend around the buckle or pass through an attachment slot. Because the buckle 40 does not have create room for inner strap 24 to loop back on itself, buckle 40 can be thinner than the buckle of the CAT GEN 7 (e.g., thickness measured from the inner surface that faces the object about which the tourniquet loops to the outer surface that faces away from the object).

Also, compared to some configurations requiring the inner band to bend around the buckle or pass through an attachment slot and then be joined (sewn/welded/etc.) in a loop, the overmolded configuration, in some embodiments, can shorten the outer sleeve tunnel section for a given amount of tightening capacity.

Further, by eliminating or reducing the need for the working segment of inner strap 24 to be woven through routing slots in the latch plate, embodiments can reduce hardware-induced friction. This reduction in hardware-induced friction facilitates smoother strap movement during windlass activation, thereby lowering the torque required to achieve effective tightening, allowing for smaller hardware components. With a smaller torque requirement, hardware such as the buckle, latch plate, and windlass can be smaller. Furthermore, reducing abrupt directional changes and pinching of the inner strap, tourniquet 10 can promote more uniform inner strap motion, which in turn mitigates sudden pressure spikes and torque surges.

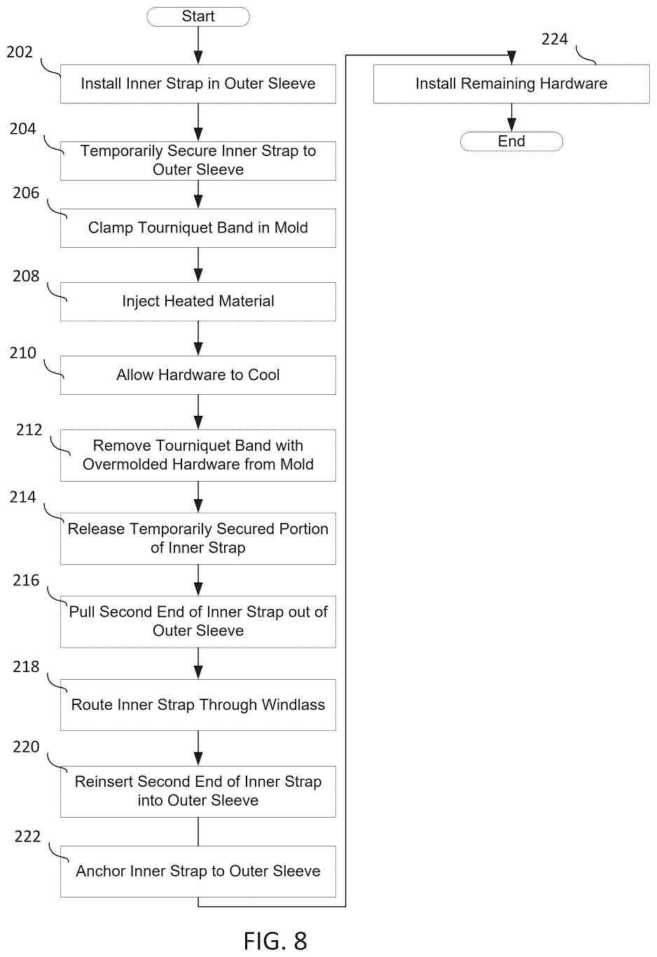

FIG. 8 is a flow chart illustrating one exemplary manufacturing method for tourniquet 10. At step 202, the inner strap 24 is installed within the outer sleeve 22. Inner strap 24 may be installed with the first end 34 of the inner strap 24 projecting out of the first end 12 of outer sleeve 22. At step 204, a portion of the inner strap away from the portion of the inner strap that will be in the mold may be temporarily stitched or otherwise affixed to a portion of outer sleeve 22 that will be away from the mold to help maintain positional stability of inner strap 24 relative to outer sleeve 22 during molding. For example, the second end 36 of the inner strap may be temporarily sewn or otherwise affixed to the tip of outer sleeve 22.

In other example embodiments, the first end of inner strap 24 is into the outer sleeve 22 via aperture 54b until it reaches its position for a subsequent molding operation. The remainder of inner strap 24 may remain outside of outer sleeve 22 and be temporarily secured to the outside of outer sleeve 22 or otherwise positioned and secured for subsequent steps.

At step 206, the terminal section of the tourniquet band 20 is then placed and clamped within one or more molds configured to form buckle 40 and any other hardware to be overmolded to the tourniquet band, such as, the latch plate 62 in some embodiments. At step 208, the heated plastic is introduced into the mold at a temperature selected to promote molecular bonding between the injected plastic and the tourniquet band materials. At step 210 the hardware is allowed to cool in the mold.

Once the injected plastic has cooled and solidified, tourniquet band 20 with the overmolded hardware is removed from the mold (step 212). At step 214, the temporarily secured portion of the inner strap 24 is then released allowing, at step 216, the second end 36 of inner strap 24 to be pulled out of outer sleeve 22. At step 218, the second end 36 of inner strap 24 is then routed through the windlass 52 and, at step 220, reinserted into the outer sleeve 22 for final assembly. At step 222, a portion of inner strap 24 is anchored to outer sleeve 22, such as by sewing, glueing, stapling, clamping, or heat/ultra-sound (sonic) welding, or another technique. At step 224, remaining hardware is installed. For example, at this stage, clip 74 may also be installed to complete the device.

FIG. 8 is merely an illustrative example, and the disclosed subject matter is not limited to the ordering or number of steps illustrated. Embodiments may implement additional steps or alternative steps, omit steps, or repeat steps. As just one example, latch plate 62 may be attached to tourniquet band 20 by sewing, glueing, stapling, clamping, or heat/ultra-sound (sonic) welding before or after buckle 40 is overmolded onto tourniquet band 20.

While embodiments described herein have been discussed primarily with reference to a single pass inner strap, an overmolded buckle or other features described herein may be used with a double-routed strap. For example, the middle (or other portion) of inner strap 24 may be affixed to outer sleeve 22 (e.g., at the tip or elsewhere on sleeve 22) and the two ends of inner strap 24 routed to the first end 12 of the outer sleeve 22. A buckle may be overmolded onto the two ends of the inner strap 24. In such an embodiment, inner strap 24 may be single or double routed through the windlass.

As another example, the inner strap 24 may be folded over on itself and the ends of the inner strap 24 affixed to outer sleeve 22 (e.g., at the tip or elsewhere on sleeve 22) or otherwise secured. A buckle may be overmolded onto the outer sleeve and the fold point of inner strap 24. In such an embodiment, inner strap 24 may be double routed through the windlass.

In another embodiment, the buckle is overmolded to the first end of outer sleeve 22 and the first ends of two inner straps (e.g., stacked together). The inner strap extends from the buckle through the windlass and is anchored to outer sleeve 22 away from the windlass. For example, the second ends of the inner straps may be anchored at the tip of outer sleeve 22. While each such inner strap may be single pass routed, multiple layers of inner strap can pass through the windlass.

While various features described herein are discussed in terms of reducing tourniquet size, features described herein may be used in larger tourniquets as well. For example, a larger tourniquet that employs single path routing may benefit from reduced torque requirements and smoother pressure application.

It will be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the meaning of “in”includes “in”and “on”unless the context clearly dictates otherwise.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” “in one embodiment.”

Thus, while the invention has been described with respect to specific embodiments thereof, these embodiments are merely illustrative, and not restrictive of the invention. Rather, the description is intended to describe illustrative embodiments, features and functions in order to provide a person of ordinary skill in the art context to understand the invention without limiting the invention to any particularly described embodiment, feature or function, including any such embodiment feature or function described. While specific embodiments of, and examples for, the invention are described herein for illustrative purposes only, various equivalent modifications are possible within the spirit and scope of the invention, as those skilled in the relevant art will recognize and appreciate.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component.

What is claimed IS:

1. A tourniquet for restricting a flow of blood in a body part, the tourniquet comprising:
a tourniquet band, the tourniquet band having a tourniquet band first end, a tourniquet band tail end, the tourniquet band comprising:
an outer sleeve, the outer sleeve having an outer sleeve first end and an outer sleeve second end; and
an inner tightening strap in slidable engagement with the outer sleeve, the inner tightening strap having an inner tightening strap first end and an inner tightening strap second end, wherein a portion of the inner tightening strap is anchored to the outer sleeve, the inner tightening strap comprising a twill weave of polymer threads;
a windlass operable to tighten the inner tightening strap, the windlass defining an aperture through which the inner tightening strap is single-routed; and
a buckle anchored to the tourniquet band first end, the buckle defining an adjustment port adapted for receiving the tourniquet band tail end for forming the tourniquet band into a loop.

2. The tourniquet of claim 1, wherein the polymer threads comprise nylon threads.

3. The tourniquet of claim 1, wherein the polymer threads comprise ultra-high-molecular-weight-polyethylene threads.

4. The tourniquet of claim 1, wherein the inner tightening strap second end is anchored to the outer sleeve.

5. The tourniquet of claim 1, wherein the aperture has a clearance percentage of 47%-75% clearance.

6. The tourniquet of claim 1, wherein the inner tightening strap is approximately 1 inch wide and 0.034 inches thick.

7. The tourniquet of claim 6, wherein the aperture has a clearance percentage of 47%-75% clearance.

8. The tourniquet of claim 1, wherein the inner tightening strap second end is anchored to the outer sleeve.

9. The tourniquet of claim 1, further comprising a plate coupled to the outer sleeve behind the windlass, the plate extending from a first end to a second end and having a side that faces the windlass, wherein the outer sleeve defines an opening at the plate through which the inner tightening strap is routed to the windlass.

10. The tourniquet of claim 9, wherein the inner tightening strap does not weave through slots in the plate between the inner tightening strap second end and the windlass and does not weave through slots in the plate between the windlass and the buckle.

11. The tourniquet of claim 10, wherein a portion of the outer sleeve extends from the first end of the plate to the second end of the plate over the side of the plate that faces the windlass.

12. The tourniquet of claim 9, wherein the plate is spaced from the buckle and is coupled to an inner side of the outer sleeve.

13. The tourniquet of claim 9, further comprising a pair of hooked catches for securing the windlass, wherein the pair of hooked catches extend outward from the plate.

14. The tourniquet of claim 13 wherein the outer sleeve and inner band run between the pair of hooked catches.

15. The tourniquet of claim 14, further comprising a clip coupled to a first hooked catch from the pair of hooked catches and configured to rotate to engage a second hooked catch from the pair of hooked catches to further secure the windlass.

16. The tourniquet of claim 15, wherein the clip comprises a metal wire form and is rotatable between an open position and a closed position, wherein the second hooked catch includes a retaining feature to lock the clip in the closed position.

17. The tourniquet of claim 1, wherein the buckle is overmolded to the outer sleeve and the inner tightening strap.

18. A tourniquet for restricting a flow of blood in a body part, the tourniquet comprising:
a tourniquet band, the tourniquet band comprising:
an outer sleeve, the outer sleeve having an outer sleeve first end and an outer sleeve second end; and
an inner tightening strap in slidable engagement with the outer sleeve, the inner tightening strap having an inner tightening strap first end and an inner tightening strap second end, wherein a portion of the inner tightening strap away from the inner tightening strap first end is anchored to the outer sleeve and wherein the inner tightening strap first end projects past the outer sleeve first end, the inner tightening strap formed of twill weave of polymer threads; a windlass operable to tighten the inner tightening strap, the windlass defining an aperture, wherein the inner tightening strap is single pass routed through the aperture, and wherein the aperture has a clearance percentage of 47%-75% clearance;
a latch plate, coupled to the tourniquet band behind the windlass, the latch plate extending from a first latch plate end to a second latch plate end and having a side that faces the windlass, wherein a portion the outer sleeve extends from the first latch plate end to the second latch plate end over the side of the latch plate that faces the windlass, wherein the inner tightening strap emerges from the outer sleeve at the latch plate and wherein the latch plate comprises at least one hooked catch for securing the windlass, wherein the outer sleeve defines an opening at the latch plate through which the inner tightening strap is routed to the windlass; and
a buckle anchored to the outer sleeve first end, wherein the buckle comprises a structural frame defining an adjustment port for receiving the outer sleeve second end for forming the tourniquet band into a loop.

19. The tourniquet of claim 18, wherein the polymer threads comprise nylon threads.

20. The tourniquet of claim 18, wherein the polymer threads comprise ultra-high-molecular-weight-polyethylene threads.
